(19) United States
(12) Patent Application Publication
(10) Pub. No.: US 2010/0292684 A1
(43) Pub. Date: Nov. 18, 2010

(54) TISSUE MODIFICATION DEVICES AND METHODS OF THE SAME

(76) Inventors: James S. Cybulski, Menlo Park, CA (US); Eric E. Schultz, Menlo Park, CA (US); Fred R. Seddiqui, Los Altos, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/467,122

(22) Filed: May 15, 2009

(51) Int. Cl. A61B 18/18; A61B 18/14

(52) U.S. Cl. 606/33; 606/41

(57) ABSTRACT

Tissue modification devices are provided. Aspects of the devices include an elongated member having a proximal end and a distal end, an tissue modifier positioned at the distal end, and a linear mechanical actuator configured to linearly translate the RF electrode relative the distal end according to a linear translation waveform. The devices may be configured to operate the tissue modifier according to an operating waveform that is synchronized with the linear translation waveform. Also provided are methods of modifying an internal target tissue of a subject using the tissue modification devices.

Traditional surgical procedures, both therapeutic and diagnostic, for pathologies located within the body can cause significant trauma to the intervening tissues. These procedures often require a long incision, extensive muscle stripping, prolonged retraction of tissues, denervation and devascularization of tissue. These procedures can require operating room time of several hours and several weeks of post-operative recovery time due to the destruction of tissue during the surgical procedure. In some cases, these invasive procedures lead to permanent scarring and pain that can be more severe than the pain leading to the surgical intervention.

The development of percutaneous procedures has yielded a major improvement in reducing recovery time and post-operative pain because minimal dissection of tissue, such as muscle tissue, is required. For example, minimally invasive surgical techniques are desirable for spinal and neurosurgical applications because of the need for access to locations within the body and the danger of damage to vital intervening tissues. While developments in minimally invasive surgery are steps in the right direction, there remains a need for further development in minimally invasive surgical instruments and methods.

### SUMMARY

Tissue modification devices are provided. Aspects of the devices include an elongated member having a proximal end and a distal end, a tissue modifier (e.g., an RF electrode) positioned at the distal end, and a linear mechanical actuator configured to linearly translate the tissue modifier relative the distal end according to a linear translation waveform. The devices may be configured to operate the tissue modifier according to an operating waveform that is synchronized with the linear translation waveform. Also provided are methods of modifying an internal target tissue of a subject using the tissue modification devices.

### BRIEF DESCRIPTION OF THE FIGURES

FIGS. 1A and B provide two different views of a tissue modification device according to an embodiment of the invention.

FIG. 2 provides a view of the distal end of a device according to one embodiment of the invention.

FIG. 3 provides various views of a linear actuator that finds use in certain embodiments of devices of the invention.

### DETAILED DESCRIPTION

Tissue modification devices are provided. Aspects of the devices include an elongated member having a proximal end and a distal end, a tissue modifier positioned at the distal end, and a linear mechanical actuator configured to linearly translate the tissue modifier relative the distal end according to a linear translation waveform. The devices may be configured to operate the tissue modifier with an operating waveform that may be synchronized with the linear translation waveform. Also provided are methods of modifying an internal target tissue of a subject using the tissue modification devices.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing various aspects of the invention, aspects of embodiments of the subject tissue modification devices are described first in greater detail. Next, embodiments of methods of modifying an internal target tissue of a subject in which the subject tissue modification devices may find use are reviewed in greater detail.

### Tissue Modification Devices

Aspects of the invention include a tissue modification device useful for modifying an internal target tissue site, e.g., a spinal location that is near or inside an intervertebral disc (IVD). Embodiments of the subject tissue modification devices include a tissue modification device comprising: an elongated member having a proximal end and a distal end; a tissue modifier positioned at the distal end; and a linear mechanical actuator configured to linearly translate the tissue modifier (e.g., an RF electrode) relative the distal end according to a linear translation waveform.

The tissue modification devices of embodiments of the invention may be dimensioned such that at least the distal end of the devices can pass through a minimally invasive body opening. As such, at least the distal end of the devices of these embodiments may be introduced to an internal target site of a patient, e.g., a spinal location that is near or inside of an intervertebral disc, through a minimal incision, e.g., one that is less than the size of an incision employed for an access device having a outer diameter of 20 mm or smaller, e.g., less than 75% the size of such an incision, such as less than 50% of the size of such an incision, or smaller. In some instances, at least the distal end of the elongated member of the devices is dimensioned to pass through a Cambin's triangle. The Cambin's triangle (also known in the art as the Pambin's triangle) is an anatomical spinal structure bounded by an exiting nerve root and a traversing nerve root and a disc. The exiting root is the root that leaves the spinal canal just cephalad (above) the disc, and the traversing root is the root that leaves the spinal canal just caudad (below) the disc. Where the distal end of the elongated member is dimensioned to pass through a Cambin's triangle, at least the distal end of the device has a longest cross-sectional dimension that is 10 mm or less, such as 8 mm or less and including 7 mm or less. In some instances, the elongated member has an outer diameter that is 7.5 mm or less, such as 7.0 mm or less, including 6.7 mm or less, such as 6.6 mm or less, 6.5 mm or less, 6.0 mm or less, 5.5 mm or less, 5.0 mm or less.

As summarized above, tissue modification devices of the systems of the invention include an elongated member. As this component of the devices is elongated, it has a length that is 1.5 times or longer than its width, such as 2 times or longer than its width, including 5 or even 10 times or longer than its width, e.g., 20 times longer than its width, 30 times longer than its width, or longer. The length of the elongated member may vary, an in some instances ranges from 5 cm to 20 cm, such as 7.5 cm to 15 cm and including 10 to 12 cm. The elongated member may have the same outer cross-sectional dimensions (e.g., diameter) along its entire length. Alternatively, the cross-sectional diameter may vary along the length of the elongated member.

The elongated members of the subject tissue modification devices have a proximal end and a distal end. The term “proximal end”, as used herein, refers to the end of the elongated member that is nearer the user (such as a physician operating the device in a tissue modification procedure), and the term “distal end”, as used herein, refers to the end of the elongated member that is nearer the internal target tissue of the subject during use. The elongated member is, in some instances, a structure of sufficient rigidity to allow the distal end to be pushed through tissue when sufficient force is applied to the proximal end of the elongate member. As such, in these embodiments the elongated member is not pliant or flexible, at least not to any significant extent.

The tissue modification devices of the invention include a distal end tissue modifier. As the tissue modifier is a distal end integrated tissue modifier, it is located at or near the distal end of the elongated member. Accordingly, it is positioned at 10 mm or closer to the distal end, such as at 5 mm or closer to the distal end, including at 2 mm or closer to the distal end. In some instances, the tissue modifier is located at the distal end of the elongated member.

Tissue modifiers are components that interact with tissue in some manner to modify the tissue in a desired way. The term modify is used broadly to refer to changing in some way, including cutting the tissue, ablating the tissue, delivering an agent(s) to the tissue, freezing the tissue, etc. As such, of interest as tissue modifiers are tissue cutters, tissue ablators, tissue freezing/heating elements, agent delivery devices, etc. Tissue cutters of interest include, but are not limited to: blades, liquid jet devices, lasers and the like. Tissue ablators of interest include, but are not limited to ablation devices, such as devices for delivery ultrasonic energy (e.g., as employed in ultrasonic ablation), devices for delivering plasma energy, devices for delivering radiofrequency (RF) energy, devices for delivering microwave energy, etc. Energy transfer devices of interest include, but are not limited to: devices for modulating the temperature of tissue, e.g., freezing or heating devices, etc.

In some instances, the tissue modifier is a distal end integrated tissue modifier. As the tissue modifier is integrated at the distal end of the device, it cannot entirely be removed from the remainder of the device without significantly compromising the structure and functionality of the device. While the tissue modifier cannot entirely be removed from the device without compromising the structure and functionality of the device, components of the tissue modifier may be removable and replaceable. For example, a RF electrode tissue modifier may be configured such that the wire component of the tissue modifier may be replaceable while the remainder of the tissue modifier is not. Accordingly, the devices of the present invention are distinguished from devices which include a “working channel” through which a separate autonomous tissue modifier device, such as autonomous RF electrode device, is passed through. In contrast to such devices, since the tissue modifier of the present device is integrated at the distal end, it is not a separate device from the elongated member that is merely present in a working channel of the elongated member and which can be removed from the working channel of such an elongated member without structurally compromising the elongated member in any way. The tissue modifier may be integrated with the distal end of the elongated member by a variety of different configurations. Integrated configurations include configurations where the tissue modifier is fixed relative to the distal end of the elongated member, as well as configurations where the tissue modifier is movable to some extent relative to the distal end of the elongated member may be employed in devices of the invention. Specific configurations of interest are further described below in connection with the figures.

In some instances, the tissue modifier is a radiofrequency (RF) electrode. In these embodiments, the tissue modification devices include a radiofrequency (RF) electrode positioned at the distal end of the elongated member. RF electrodes are devices for the delivery of radiofrequency energy, such as ultrasound, microwaves, and the like. In some instances, the RF electrode is an electrical conductor for delivering RF energy to a particular location, such as a desired target tissue. For instance, in certain cases, the RF electrode can be a RF ablation electrode. RF electrodes of the subject tissue modification devices can include a conductor, such as a metal wire, and can be dimensioned to access an intervertebral disc space. For example, RF electrodes can have a configuration (such as a U-shaped configuration or other desirable configuration) which has a longest cross-sectional dimension that is 10 mm or less, such as 8 mm or less and including 7 mm or less.

As reviewed above, devices of the invention may include a linear mechanical actuator for linearly translating a distal end element of the device, such as the tissue modifier (e.g., a RF electrode) relative to the distal end of the elongate member. By “linearly translating” is meant moving the tissue modifier along a substantially straight path. As used herein, the term “linear” also encompasses movement of the tissue modifier in a non-straight (i.e., curved) path. For instance, the path of movement of the tissue modifier can be deflected from a substantially straight path if the electrode encounters a tissue of a different density (such as, cartilage, bone, etc.), or if the conformation of the tissue the electrode is passing through is not straight, etc.

When actuated by a linear mechanical actuator, the tissue modifier is cyclically displaced from a “neutral” position along its axial extension to positions displaced distally or proximally from the neutral position, with maximum displacement from the neutral position corresponding to the vibratory amplitude. Thus, the linear mechanical actuator actuates the tissue modifier through a distance equal to twice the vibratory amplitude and ranging from a distal extreme position to a proximal extreme position. In certain embodiments, the tissue modifier can be extended by the linear mechanical actuator from the distal end of the elongated member by 0.1 mm or more, such as 0.5 mm or more, including 1 mm or more, for instance 2 mm or more, such as 5 mm or more.

This back and forth movement of the tissue modifier relative to the distal end of the elongated member that is implemented by the linear mechanical actuator is described herein in terms of linear translation waveform. The term “linear translation waveform” as used herein refers collectively to the various parameters of the movement profile, such as frequency and displacement distance, of the tissue modifier during linear translation as implemented by the linear mechanical actuator. In some instances, the waveform may be represented as a graphical equation of frequency and displacement distance, as desired.

It is noted that the above described distal and proximal extreme positions refer to those positions implemented solely by the linear mechanical actuator. In some embodiments, the linear mechanical actuator may be the only means for translating the electrode. In other embodiments, e.g., as described in greater detail below, the linear mechanical actuator may provide vibratory amplitude that is superimposed on another independent control over electrode translation which moves the electrode over a distance significantly greater than the vibratory amplitude, e.g. 10 mm or more, such as 20 mm or more, including 30 mm or more, for instance 40 mm or more. In this case, the tissue modifier may be extended beyond the range defined by the above described linear mechanical actuator distal and proximal extreme positions. For example, a manual control (e.g., a thumbwheel or analogous structure) may be provided on the device which permits a user to move the tissue modifier relative to the distal end in a movement that is distinct from that provided by the linear mechanical actuator.

Accordingly, devices of the invention may include a linear mechanical actuator configured to linearly translate the tissue modifier relative to the distal end according to a linear translation waveform that has a linear translation frequency. The linear mechanical actuator can be any of a variety of actuators convenient for use in the subject devices for linearly translating the tissue modifier relative to the distal end of the elongated member. For instance, the linear mechanical actuator can be a voice coil motor (VCM), solenoid, pneumatic actuator, electric motor, etc. The linear mechanical actuator is operatively coupled to the tissue modifier. By “operatively coupled” is meant that the linear mechanical actuator is connected to the tissue modifier such that linear movement by the actuator is transferred to the tissue modifier thereby extending the tissue modifier from the distal end of the elongated member or retracting the tissue modifier towards the distal end of the elongated member depending on the direction of movement by the linear actuator.

The linear actuator provides for linear translation of the tissue modifier according to a linear translation waveform that has a linear translation frequency. In some instances, the linear translation frequency is 10 Hz or greater, such as 25 Hz or greater, including 50 Hz or greater, such as 100 Hz or greater. In some embodiments, the linear translation frequency is 70 Hz. In certain cases, the translation of the tissue modifier between the distal and proximal extreme positions occurs with a predetermined linear translation frequency while in other embodiments the linear translation frequency may not be predetermined. The translation frequency (whether or not predetermined) may depend on various factors, such as but not limited to, the type of tissue being modified, the amount of tissue being modified, the location of the tissue, the proximity of surrounding tissues, the conformation of the tissue, the type of procedure being performed, the nature of the linear mechanical actuator, the DC voltage applied to the actuator, the amplitude of the AC voltage applied to the actuator, etc.

In certain embodiments, the linear translation waveform is definable as a standard waveform, such as a sine waveform. In some cases, the sine waveform is a Hz sine waveform, such that the linear translation frequency ranges from 1 Hz to 500 Hz, such as from 1 Hz to 250 Hz, and including from 10 Hz to 100 Hz. In other cases, the linear translation waveform is definable as a non-standard, complex, or irregular waveform, or the like. For example, the linear translation waveform can be definable as a waveform comprising periods that have varying frequencies, a waveform comprising periods that have varying amplitudes, a waveform comprising periods that have varying frequencies and varying amplitudes, a superposition of two or more waveforms, and the like.

In some embodiments, the tissue modification device is configured to synchronize the linear mechanical actuation with a tissue modification device operational waveform, such as a modulated RF waveform. By “synchronize” is meant that two or more events are timed to operate in a coordinated manner. For example, two or more waveforms can be timed to operate in a coordinated manner. In some embodiments, the modulation frequency equals the linear translation frequency, and the modulation waveform is phase-shifted relative to the linear translation waveform. Synchronization of these waveforms may be achieved using a variety of different protocols and may implement one or more controllers of different formats, including hardware, software, and combinations thereof. For instance, a single common controller may generate two waveforms that are phase-shifted; alternatively, separate controllers can be arranged in a master-slave configuration to generate two waveforms that are phase-shifted; alternatively, one controller can generate a waveform, hardware (e.g., an opto-electronic encoder, a mechanical encoder, a hall sensor, or the like) can be used to trigger on a physical embodiment (such as mechanical rotation) of that waveform, and a second controller can generate a second waveform with adjustable phase shift from the trigger signal. The phase shift of the modulation waveform relative to the linear translation waveform can be positive (phase lead) or negative (phase lag), and can have a magnitude of 0° to 360° or more, such as 0° to 180°, including 60° to 120°. In certain embodiments of the invention, the modulation waveform lags the linear translation waveform by 90°.

As discussed above, the tissue modifier (e.g., a RF electrode) has distal and proximal extreme positions of its cyclic linear translation. In certain embodiments, the tissue modifier is configured to deliver RF energy to an internal target tissue while at a position other than the distal extreme position. Thus, in these cases, the modulation waveform is synchronized with the linear translation waveform such that the tissue modifier is energized when the tissue modifier is at a position other than the distal extreme position, such as while the tissue modifier is at or near the proximal extreme position. For example, as discussed above, the modulating waveform may be phase-shifted relative to the linear translation waveform.

Cyclic linear translation of the tissue modification device can facilitate a variety of functions with multiple benefits. For instance, cyclic linear translation of the tissue modifier at a fast rate relative to manually controlled translation (e.g., at a frequency greater than 10 Hz) will tend to physically advance the tissue modifier into soft tissue due to the compliance of the soft tissue, while hard tissue will resist deformation and will thus not allow the tissue modifier to physically advance into the hard tissue. Consequently, the electrode will push back against the elongated body as it encounters hard tissue, thus producing tactile feedback to the user. In some embodiments, synchronization of the tissue modifier's modulation waveform with its linear translation waveform provides additional benefits. For instance, rapid retraction of the electrode from hard tissue that it encounters will leave the tissue modifier physically separated from the hard tissue by a gap as the tissue modifier approaches the proximal extreme position. In some embodiments, the tissue modifier tip is activated only when the tissue modifier is at or near the proximal extreme position, as mentioned above. This has the effect of preferentially delivering the tissue modification energy to soft, compliant tissue as opposed to hard, stiff tissue. Stated otherwise, this provides tissue discrimination based on elastic modulus. In the case of spinal surgery applications requiring removal of nuclear material, such as fusion, total disc replacement, and partial disc replacement, synchronization of the modulation waveform with the linear translation waveform facilitates the delivery of tissue modification energy to the nucleus pulposus (soft, compliant tissue) while minimizing the delivery of tissue modification energy to the disc annulus (hard, stiff tissue) and the endplates of the vertebral bodies (hard, stiff tissue). In addition, cyclic linear translation of the tissue modifier helps to prevent a condition where the electrode sticks to tissue as it ablates it, resulting in increased thermal effects to the surrounding tissue, ineffective or discontinuous tissue dissection, buildup of charred or otherwise modified tissue on the tissue modifier tip, or a combination thereof. Additionally, cyclic linear translation of the tissue modifier helps chop the dissected tissue into smaller pieces, thus facilitating aspiration of the dissected tissue. Finally, the rapid movement inherent in the cyclic translation of an RF electrode tissue modifier can facilitate the generation of a plasma corona at the distal tip of the RF electrode at lower RF voltage and power settings. In some cases, the rapid cyclic translation of the RF electrode induces cavitation in the surrounding saline solution, which effectively reduces the amount of saline that must be vaporized to strike the plasma, thus lowering the minimum RF power setting required to generate the plasma corona.

In some instances, the tissue modifier is supplied with current from an RF energy source. The voltage signal driving the current to the tissue modifier may be definable as a sine, square, saw-tooth, triangle, pulse, non-standard, complex, or irregular waveform, or the like, with a well-defined operating frequency. For example, the operating frequency can range from 1 KHz to 50 MHz, such as from 100 KHz to 25 MHz, and including from 250 KHz to 10 MHz. In some embodiments, the RF voltage signal is a sine wave with operating frequency 460 kHz. Furthermore, the tissue modifier's operating frequency can be modulated by a modulation waveform. By “modulated” is meant attenuated in amplitude by a second waveform, such as a periodic signal waveform. The modulation waveform may be definable as a sine, square, saw-tooth, triangle, pulse, non-standard, complex, or irregular waveform, or the like, with a well-defined modulation frequency. For example, the modulation frequency can range from 1 Hz to 10 kHz, such as from 1 Hz to 500 Hz, and including from 10 Hz to 100 Hz. In some embodiments, the modulation waveform is a square wave with modulation frequency 70 Hz.

In some embodiments, a RF tuner is included as part of the RF energy source. The RF tuner includes basic electrical elements (e.g., capacitors and inductors) which serve to tailor the output impedance of the RF energy source. The term “tailor” is intended here to have a broad interpretation, including affecting an electrical response that achieves maximum power delivery, affecting an electrical response that achieves constant power (or voltage) level under different loading conditions, affecting an electrical response that achieves different power (or voltage) levels under different loading conditions, etc. Furthermore, the elements of the RF tuner can be chosen so that the output impedance is dynamically tailored, meaning the RF tuner self-adjusts according to the load impedance encountered at the electrode tip. For instance, the elements may be selected so that the electrode has adequate voltage to develop a plasma corona when the electrode is placed in a saline solution (with saline solution grounded to return electrode), but then may self-adjust the voltage level to a lower threshold when the electrode contacts tissue (with tissue also grounded to return electrode, for example through the saline solution), thus dynamically maintaining the plasma corona at the electrode tip while minimizing the power delivered to the tissue and the thermal impact to surrounding tissue. RF tuners, when present, can provide a number of advantages. For example, delivering RF energy to target tissue through the distal tip of the electrode is challenging since RF energy experiences attenuation and reflection along the length of the conductive path from the RF energy source to the electrode tip, which can result in insertion loss. Inclusion of a RF tuner, e.g., as described above, can help to minimize and control insertion loss.

In some instances, the devices include one or more sensors configured to obtain linear translation data. By linear translation data is meant information about the linear translation of the RF electrode, where such information may include information about the direction of translation, velocity of translation, acceleration/deceleration of translation, etc. The sensor or sensors, when present, may be positioned at any convenient location of the elongate member, e.g., at the distal end, etc., so long as the sensor or sensors are positioned so that the desired linear translation data may be obtained. Any of a variety of different types of sensors may be employed, where sensors of interest include, but are not limited to: optical encoders, mechanical encoders, optoelectronic sensors, Hall effect sensors, position sensors, motion detection sensors, and the like.

When sensors are present such that linear translation data may be obtained, the linear translation data may be employed by the device to synchronize the operating and linear translation waveforms. As such, in some instances the controllers are configured to synchronize the linear translation waveform and operating waveform based on the linear translation waveform data obtained by the sensor or sensors of the device. In other words, the linear translation data is used by the controller in some way to synchronize the operating waveform the linear translation waveform.

Where desired tissue modification devices of the invention may be configured for distal end steerability. By “distal end steerability” is meant the ability to maneuver or orient the distal end of the device as desired during a procedure, e.g., by using controls positioned at the proximal end of the device. In these embodiments, the devices include a steerability mechanism (or one or more elements located at the distal end, such as the RF electrode, etc) which renders the distal end maneuverable as desired through proximal end control. The term “steerability”, as used herein, refers to a mechanism that provides a user steering functionality, such as the ability to change direction in a desired manner, such as by moving left, right, up or down relative to the initial direction. The steering functionality can be provided by a variety of different mechanisms. Examples of suitable mechanisms include, but are not limited to one or more wires, plates, tubes, wire meshes, etc. Steerability mechanisms of interest are further described in pending U.S. Provisional Patent Application Ser. No. 61/082,774; as well as published PCT Application Publication Nos. WO 2009029639; WO 2008/094444; WO 2008/094439 and WO 2008/094436; the disclosures of which are herein incorporated by reference.

Certain embodiments of the subject devices include a visualization element integrated at the distal end of the tissue modification device, e.g., near to or part of the tissue modification element. Of interest as visualization elements are imaging sensors. Imaging sensors of interest are miniature in size so as to be integrated with the tissue modification device at the distal end. The visualization element may be integrated with a component of interest, e.g. the elongated member of the tissue modification device. As the visualization element(s) is integrated at the distal end of the device, it cannot be removed from the remainder of the device without significantly compromising the structure of device. Miniature imaging sensors of interest are those that, when integrated at the distal end of an elongated structure along with an illumination source, e.g., such as an LED or fiber optic light source, can be positioned on a probe having a minimal cross section dimension to facilitate access to the intervertebral disc space. In certain embodiments, the miniature imaging sensors have a cross-sectional area that is sufficiently small for its intended use and yet retain a sufficiently high matrix resolution. Imaging sensors of interest are those that include a photosensitive component, e.g., array of photosensitive elements, coupled to an integrated circuit, where the integrated circuit is configured to obtain and integrate the signals from the photosensitive array and output the analog data to a backend processor. The image sensors of interest may be viewed as integrated circuit image sensors, and include complementary metal-oxide-semiconductor (CMOS) sensors and charge-coupled device (CCD) sensors. The image sensors may further include a lens positioned relative to the photosensitive component so as to focus images on the photosensitive component. A signal conductor may be present to connect the image sensor at the distal end to a device at the proximal end of the elongate member, e.g. in the form of one or more wires running along the length of the elongate member from the distal to the proximal end.

Imaging sensors of interest may be either frontside or backside illumination sensors, and have sufficiently small dimensions while maintaining sufficient functionality to be integrated at the distal end of the elongated members of the devices of the invention. Aspects of these sensors are further described in one or more the following U.S. patents, the disclosures of which are herein incorporated by reference: U.S. Pat. Nos. 7,388,242; 7,368,772; 7,355,228; 7,345,330; 7,344,910; 7,268,335; 7,209,601; 7,196,314; 7,193,198; 7,161,130; and 7,154,137.

In certain embodiments, devices of the invention may further include an irrigator and aspirator configured to flush an internal target tissue site. As such, the elongated member may further include one or more lumens that run at least the substantial length of the device, e.g. for performing a variety of different functions. In certain embodiments where it is desired to flush (i.e., wash) the target tissue site at the distal end of the elongated member (e.g. to remove ablated tissue from the location, etc.), the elongated member may include both an irrigation lumen and an aspiration lumen. Thus, the tissue modification device can comprise an irrigation lumen located at the distal end of the elongated member, and the tissue modification device can comprise an aspiration lumen located at the distal end of the elongated member. During use, the irrigation lumen is operatively connected to a fluid source (e.g., a physiologically acceptable fluid, such as saline) at the proximal end of the device, where the fluid source is configured to introduce fluid into the lumen under positive pressure, e.g., at a pressure ranging from 0 psi to 60 psi, so that fluid is conveyed along the irrigation lumen and out the distal end. While the dimensions of the irrigation lumen may vary, in certain embodiments the longest cross-sectional dimension of the irrigation lumen ranges from 0.5 mm to 5 mm, such as 1 mm to 4 mm, including 1 mm to 3 mm. During use, the aspiration lumen is operatively connected to a source of negative pressure (e.g., a vacuum source) at the proximal end of the device. While the dimensions of the aspiration lumen may vary, in certain embodiments the longest cross-sectional dimension of the aspiration lumen ranges from 1 mm to 7 mm, such as 2 mm to 6 mm, including 3 mm to 5 mm. In some instances, the negative pressure source is configured to draw fluid and/or tissue from the target tissue site at the distal end into the aspiration lumen under negative pressure, e.g., at a negative pressure ranging from 300 to 600 mmHg, such as 550 mmHg, so that fluid and/or tissue is removed from the tissue site and conveyed along the aspiration lumen and out the proximal end, e.g., into a waste reservoir. In certain embodiments, the irrigation lumen and aspiration lumen may be separate lumens, while in other embodiments, the irrigation lumen and the aspiration lumen can be included in a single lumen, for example as concentric tubes with the inner tube providing for aspiration and the outer tube providing for irrigation.

Where desired, the devices may include a control structure, such as a handle, operably connected to the proximal end of the elongated member. By “operably connected” is meant that one structure is in communication (for example, mechanical, electrical, optical connection, or the like) with another structure. When present, the control structure (e.g., handle) is located at the proximal end of the device. The handle may have any convenient configuration, such as a hand-held wand with one or more control buttons, as a hand-held gun with a trigger, etc., where examples of suitable handle configurations are further provided below.

In some embodiments, the distal end of the elongated member is rotatable about its longitudinal axis when a significant portion of the operating handle is maintained in a fixed position. As such, at least the distal end of the elongated member can turn by some degree while the handle attached to the proximal end of the elongated member stays in a fixed position. The degree of rotation in a given device may vary, and may range from 0 to 3600, such as 0 to 2700, including 0 to 1800.

Devices of the invention may be disposable or reusable. As such, devices of the invention may be entirely reusable (e.g., be multi-use devices) or be entirely disposable (e.g., where all components of the device are single-use). In some instances, the device can be entirely reposable (e.g., where all components can be reused a limited number of times). Each of the components of the device may individually be single-use, of limited reusability, or indefinitely reusable, resulting in an overall device or system comprised of components having differing usability parameters.

Devices of the invention may be fabricated using any convenient materials or combination thereof, including but not limited to: metallic materials such as tungsten, stainless steel alloys, platinum or its alloys, titanium or its alloys, molybdenum or its alloys, and nickel or its alloys, etc; polymeric materials, such as polytetrafluoroethylene, polyimide, PEEK, and the like; ceramics, such as alumina (e.g., STEATITE™ alumina, MAECOR™ alumina), etc.

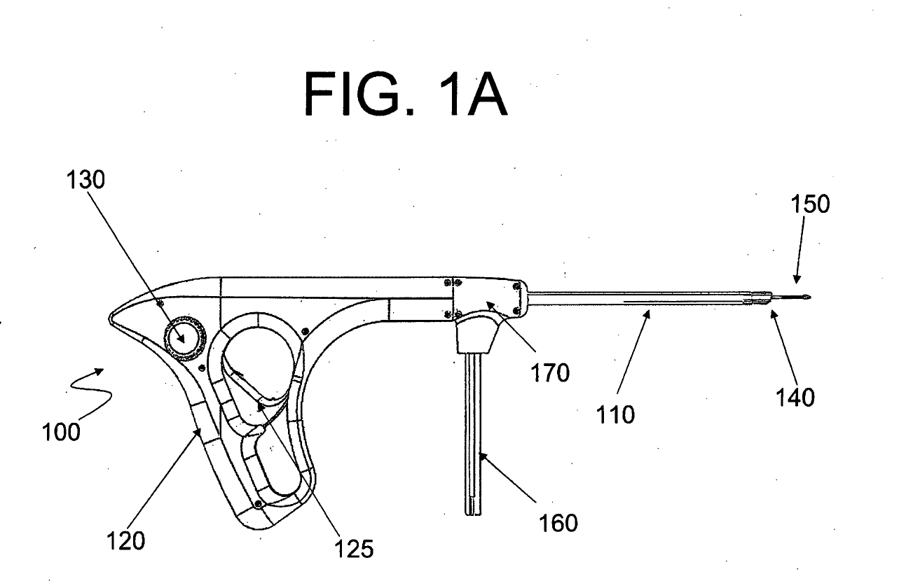

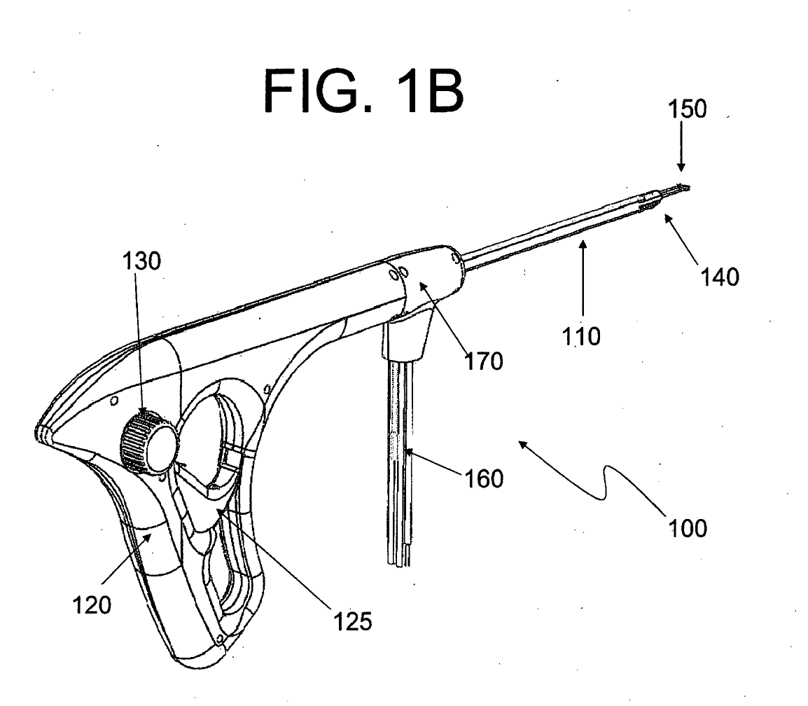

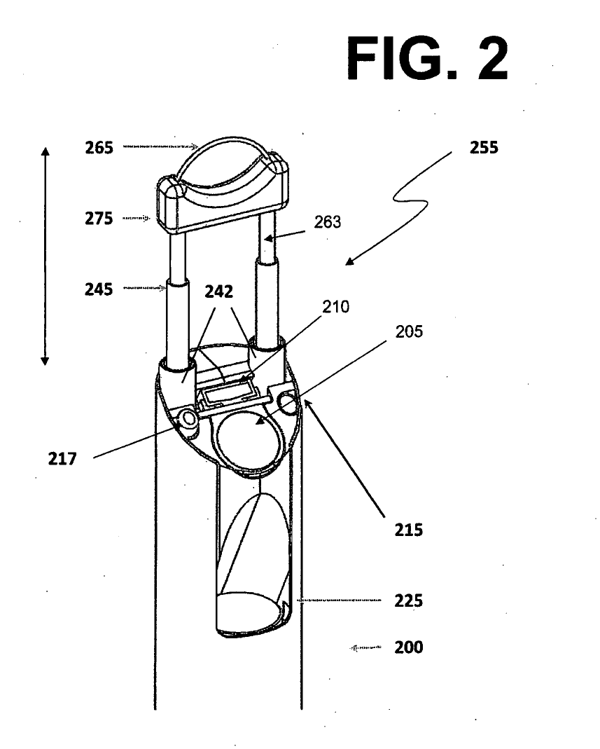

Various aspects of device embodiments of the invention have been described in varying detail above. Device embodiments will now be described in further detail in terms of figures. FIGS. 1A and 1B provide two different side views of a device 100 according to one embodiment of the invention. Device 100 includes an elongated member 110 and an operating handle 120 at the proximal end of the elongated member 110. The operating handle has a gun configuration and includes a trigger 125 and thumbwheel 130 which provide a user with manual operation over certain functions of the device, e.g., RF electrode positioning and extension. Located at the distal end of the elongated member is an integrated visualization sensor 140 and tissue modifier 150. Control elements 160 (which may include aspiration and irrigation lumens, control/power wires, etc.) exit the handle 120 at the distal end region 170, which region 170 is rotatable relative to the remainder of the handle 120. A variety of additional components may be present at the distal end of the elongated member, which additional elements may include irrigators, aspirators, articulation mechanisms, etc. as described generally above. More details regarding the distal end of elongate member 140 may be seen in FIG. 2.

FIG. 2 provides a three-dimensional view of one embodiment of a distal end of tissue modification device 200 (having a 6.5 mm outer dimension) of the invention. In FIG. 2, the distal end of the device includes and integrated circular CMOS visualization sensor 205 and integrated LED 210. Also shown is a first forward facing irrigation lumen 215 and a second irrigation lumen 217 which is slightly extended from the distal end and is side facing so that fluid emitted from lumen 217 is flowed across CMOS visualization sensor 205 to clean the sensor of debris, when needed. Also shown is an aspiration lumen 225 positioned proximal the irrigation lumens 215 and 217 and integrated CMOS visualization sensor 205, where the aspiration lumen 225 is configured to aspirate fluid and tissue debris from a target tissue site during use. The distal end further includes an integrated steerable RF electrode assembly 255. RF electrode assembly 255 includes NITINOL shape-memory guide tubes 245 extending from insulated (e.g., RF shielded) guide lumens 242. The RF electrode further includes a tungsten cutting wire 265 joined at each end to a NITINOL shape memory electrode wire 263 by a ceramic arc stop 275. As shown, the diameter of the cutting wire 265 is smaller than the diameter of the electrode wires 263, where the difference in size may vary and may range from 100 to 500 μm, such as 300 to 400 μm. RF electrode assembly may be linearly translated relative to the distal end of the elongated member as represented by the double arrow.

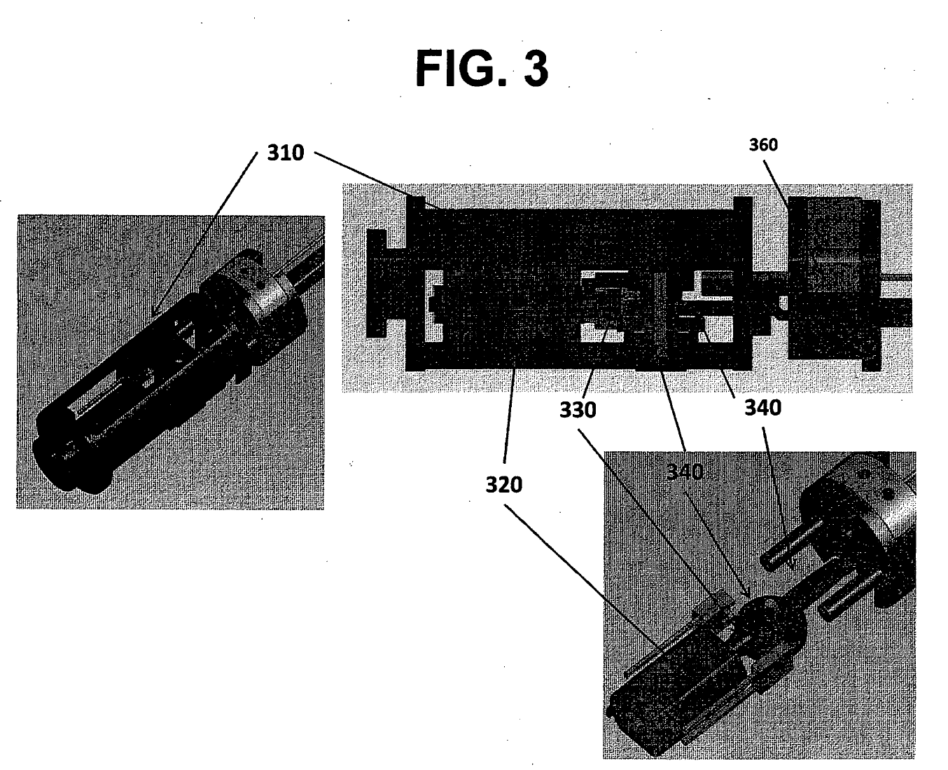

FIG. 3 provides various views of an electric motor linear actuator that may be present in devices of the invention to provide for linear translation of the RF electrode. As shown in FIG. 3, the device includes a motor carriage 310 that houses an electric motor 320. Electric motor 320 includes small bevel gear 330 which is in operative relationship with large bevel gear 340. Large bevel gear 340 is, in turn, operatively connected to cam follower 350 which is operatively connected to the RF electrode at transmission point 360.

Aspects of the subject invention include tissue modification systems, where the systems include a tissue modification device connected to one or more extra-corporeal units, e.g., a video display, control unit, etc. Systems of the invention may include a number of additional components in addition to the tissue modification devices as described above. Additional components may include access devices; root retractors; device fixation devices; image display units (such as monitors, printers, and the like); data processors, e.g., in the form of computers; data storage devices, e.g., floppy disks, hard drives, CD-ROM, DVD, flash memory, and the like; etc.

### Methods

Aspects of the subject invention also include methods of modifying an internal target tissue of a subject. Methods of the invention may include positioning the distal end of a tissue modification device of the invention in operative relationship to the internal target tissue; and then contacting the tissue modifier with the target tissue, e.g., by delivering RF energy to the internal target tissue, in a manner effective to modify the internal target tissue of the subject, as desired. As such, where the tissue modifier is a RF electrode, methods of the invention include methods of delivering RF energy to an internal target tissue location or locations. In certain of these embodiments, the method includes linearly actuating the RF electrode and synchronizing the RF electrode's operating waveform with the linear translation waveform. As described in detail above, the operating and linear translation waveforms can be phase shifted relative to each other. In certain instances, the phase shift is adjusted so that the RF electrode delivers RF energy to an internal target tissue while at a position near the extreme proximal position. This may, for example, facilitate the delivery of RF energy to the internal target tissue while minimizing the undesired delivery of RF energy to surrounding tissue. Alternatively, the device employed in the methods may be pre-synchronized. By “pre-synchronized” is meant that the timing of the predetermined linear translation waveform with respect to the RF electrode's operating waveform is configured prior to using the device. For example, the timing of a linear translation waveform with respect to the RF electrode's operating waveform can be programmed by a user, selected from various preset configurations, or selected automatically by the controller depending on the type of procedure being performed by the user.

In addition to modifying one or more internal target tissue locations, the methods of the invention may include visualizing the target tissue location, e.g., with an integrated visualization sensor on the device. In yet other embodiments, the methods may include flushing an internal target tissue site.

The internal target tissue site may vary widely. Internal target tissue sites of interest include, but are not limited to, cardiac locations, vascular locations, central nervous system locations, etc. In certain cases, the internal target tissue site comprises spinal tissue. Regarding spinal surgeries, such as total disc replacement or partial disc replacement, the present method can be performed in a manner that facilitates the delivery of RF energy to the nucleus pulposus while minimizing the undesired delivery of RF energy to the surrounding disc annulus. Thus, in some instances, the method is a method for removing nucleus pulposus tissue from a herniated intervertebral disc. In certain embodiments, the method includes inserting the elongated member of the tissue modification device into the intervertebral disc space with a field of view oriented in a first direction. To reach areas directly in front of the distal end of the elongated member, the method includes linearly extending and retracting the RF electrode with respect to the distal end of the elongated member.

The subject methods are suitable for use with a variety of mammals. Mammals of interest include, but are not limited to: race animals, e.g. horses, dogs, etc., work animals, e.g. horses, oxen etc., and humans. In some embodiments, the mammals on which the subject methods are practiced are humans.

### Utility

The subject tissue modification devices and methods find use in a variety of different applications where it is desirable to modify an internal target tissue of a subject while minimizing damage to the surrounding tissue. The subject devices and methods find use in many applications, such as but not limited to surgical procedures, where a variety of different types of tissues may be removed, including but not limited to: soft tissue, cartilage, bone, ligament, etc. Specific procedures of interest include, but are not limited to, spinal fusion, total disc replacement (TDR), partial disc replacement (PDR), procedures in which all or part of the nucleus pulposus is removed from the intervertebral disc (IVD) space, arthroplasty, and the like. As such, methods of the invention also include treatment methods, e.g., where a disc is modified in some manner to treat an existing medical condition. Treatment methods of interest include, but are not limited to: annulotomy, nucleotomy, discectomy, annulus replacement, nucleus replacement, and decompression due to a bulging or extruded disc. Additional methods in which the imaging devices find use include those described in United States Published Application No. 20080255563.

In certain embodiments, the subject devices and methods facilitate the dissection of the nucleus pulposus while minimizing thermal damage to the surrounding tissue. In addition, the subject devices and methods can facilitate the surgeon's accessibility to the entire region interior to the outer shell, or annulus, of the IVD, while minimizing the risk of cutting or otherwise causing damage to the annulus or other adjacent structures (such as nerve roots) in the process of dissecting and removing the nucleus pulposus.

Furthermore, the subject devices and methods may find use in other procedures, such as but not limited to ablation procedures, including high-intensity focused ultrasound (HIFU) surgical ablation, cardiac tissue ablation, neoplastic tissue ablation (e.g. carcinoma tissue ablation, sarcoma tissue ablation, etc.), microwave ablation procedures, and the like.

As reviewed above, devices of the invention provide variable tactile feedback depending on tissue type. In other words, devices of invention may provide different sensations to an operator, such as a surgeon, during use depending on the nature of the tissue with the distal end of the device is in contact. As such, devices and methods of the invention also find use in tissue discrimination applications, where the devices are employed to determine the particular nature of the internal tissue with which the distal end of the device is in contact, e.g., whether the distal end of the device is in contact with soft tissue, cartilage, bone, etc.

### Kits

Also provided are kits for use in practicing the subject methods, where the kits may include one or more of the above devices, and/or components of the subject systems, as described above. The kit may further include other components, e.g., guidewires, access devices, fluid sources, etc., which may find use in practicing the subject methods. Various components may be packaged as desired, e.g., together or separately.

In addition to above mentioned components, the subject kits may further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular embodiments only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A tissue modification device comprising:
an elongated member having a proximal end and a distal end;
a tissue modifier positioned at the distal end; and
a linear mechanical actuator configured to linearly translate the tissue modifier relative the distal end according to a linear translation waveform.

2. The tissue modification device according to claim 1, wherein the device further synchronizes the linear translation waveform with a tissue modifier operating waveform.

3. The tissue modification device according to claim 2, wherein the tissue modifier operating waveform is phase shifted relative to the linear translation waveform.

4. The tissue modification device according to claim 1, wherein the linear translation waveform has a frequency that is 10 Hz or greater.

5. The tissue modification device according to claim 1, wherein the tissue modifier is a RF electrode.

6. The tissue modification device according to claim 5, wherein the RF electrode has an operating waveform that is definable as KHz waveform modulated with an Hz waveform.

7. The tissue modification device according to claim 1, wherein the device comprises a sensor configured to obtain linear translation waveform data.

8. The tissue modification device according to claim 7, wherein the obtained linear translation waveform data is used to synchronize an operating waveform of the tissue modifier with the linear translation waveform.

9. The tissue modification device according to claim 7, wherein the sensor is a sensor selected from the group consisting of an optical encoder, mechanical encoder, optoelectronic sensor, and Hall effect sensor.

10. The tissue modification device according to claim 1, wherein the linear mechanical actuator is selected from a voice-coil, a solenoid and an electric motor.

11. The tissue modification device according to claim 1, wherein the device further comprises a distal end visualization sensor.

12. The tissue modification device according to claim 11, wherein the visualization sensor is selected from a CCD and CMOS sensor.

13. The tissue modification device according to claim 1, wherein the device is configured for distal end steerability.

14. The tissue modification device according to claim 1, wherein the device further comprises a distal end irrigator and aspirator.

15. The tissue modification device according to claim 1, wherein the distal end of the elongated member is dimensioned to access an intervertebral disc.

16. A method of modifying an internal target tissue of a subject, the method comprising:
(a) positioning a tissue modification device comprising:
(i) an elongated member having a proximal end and a distal end;
(ii) a tissue modifier positioned at the distal end; and
(iii) a linear mechanical actuator configured to linearly translate the tissue modifier relative the distal end according to a linear translation waveform;
in operative relationship to the internal target tissue; and
(b) modifying the internal target tissue with the tissue modifier.

17. the method according to claim 16, wherein the tissue modifier comprises a RF electrode and the method comprises delivering RF energy to the internal target tissue in a manner effective to modify the internal target tissue of the subject.

18. The method according to claim 16, wherein the method comprises operating the linear translation actuator at a frequency of 10 Hz or greater.

19. The method according to claim 17, wherein the energy delivered by the tissue modification device is modulated by a modulation waveform that is synchronized with the linear translation waveform to provide the tissue modifier with capability for tissue discrimination based on elastic modulus.

20. The method according to claim 16, wherein the tissue modification device further comprises a visualization element located at the distal end of the elongated member, and the method comprises visualizing the internal target tissue site.

21. The method according to claim 16, wherein the internal target tissue comprises spinal tissue.

22. The method according to claim 16, wherein the method further comprises discriminating between internal tissue types.
